(19) United States
(12) Patent Application Publication
(10) Pub. No.: US 2025/0049454 A1
(43) Pub. Date: Feb. 13, 2025

(54) VIBRATIONAL AND ROTATIONAL CALCULUS BASKET

(71) Applicant: GYRUS ACMI, INC D/B/A OLYMPUS SURGICAL TECHNOLOGIES AMERICA, Westborough, MA (US)

(72) Inventors: Aseem Singh, Northborough, MA (US); Kamaldeep Singh, Marlborough, MA (US)

(21) Appl. No.: 18/799,764

(22) Filed: Aug. 9, 2024

Related U.S. Application Data
(60) Provisional application No. 63/518,510, filed on Aug. 9, 2023.

(51) Int. Cl. A61B 17/22; A61B 17/00; A61B 17/221

(52) U.S. Cl. CPC A61B 17/2202; A61B 17/221; A61B 2017/00398; A61B 2017/00477; A61B 2017/00836; A61B 2017/00867; A61B 2017/22028; A61B 2017/2212

(57) ABSTRACT

Various embodiments disclosed relate to a lithotripsy device. A device may include a longitudinal shaft extending between a proximal portion and distal portion. A device may include a basket extending along the distal portion of the longitudinal shaft, the basket configured to catch a target calculus. A device may include a rotational driver operably coupled to the longitudinal shaft and the basket, the rotational driver actuatable for rotating the longitudinal shaft and the basket, wherein at least one of the longitudinal shaft and the basket is configured to vibrate in response to an acoustic energy input.

### CROSS-REFERENCE TO RELATED APPLICATION

This application claims the benefit of priority to U.S. Provisional Patent Application Ser. No. 63/518,510 entitled “VIBRATIONAL AND ROTATIONAL CALCULUS BASKET,” filed Aug. 9, 2023, the disclosure of which is incorporated herein in its entirety by reference.

### BACKGROUND

Medical endoscopes are used to inspect and perform procedures inside the human body. Such endoscopes can have, for example, a distal end with an optical or imaging system, and a proximal end with a control for device manipulation, connected through a longitudinal shaft. Such endoscopes can additionally include one or more channels such as for the provision of other medical devices to be passed into the human body for various procedures.

A variety of such devices have been developed in urology, such as various tools and methods for treatment and removal of calculi. For example, such devices can be used to break up calculi in places such as bile ducts, urinary tracts, kidneys, gall bladder, and elsewhere. Such calculi or “calculi” can be painful to a patient and may block or inhibit these various regions. Many of these calculi can be removed through breakdown, such as by ablation or other techniques. Some techniques can include acoustic lithotripsy, pneumatic lithotripsy, electro-hydraulic lithotripsy (EHL), and laser lithotripsy. In some cases, such calculi additionally should be dislodged, such as with minimal surgical intervention.

### SUMMARY OF THE DISCLOSURE

In an example, the techniques described herein relate to a lithotripsy device including: a longitudinal shaft extending between a proximal portion and distal portion; a basket extending along the distal portion of the longitudinal shaft, the basket configured to catch a target calculus; and a rotational driver operably coupled to the longitudinal shaft and the basket, the rotational driver actuatable for rotating the longitudinal shaft and the basket, wherein at least one of the longitudinal shaft and the basket is configured to vibrate in response to an acoustic energy input.

In an example, the techniques described herein relate to an ultrasonic probe including: a longitudinal shaft extending between a proximal portion and distal portion; a basket extending along the distal portion of the longitudinal shaft, the basket configured to catch a target calculus; a rotational driver operably coupled to the longitudinal shaft and the basket, the rotational driver actuatable for rotating the longitudinal shaft and the basket; and a transducer acoustically coupled to at least one of the longitudinal shaft or the basket, the transducer configured to produce an ultrasonic excitation, wherein the basket is configured to vibrate in response to an acoustic energy input.

In an example, the techniques described herein relate to a method of removing a calculus, the method including: dislodging one or more calculi or calculi fragments with a basket, wherein dislodging includes vibrating the basket according to an acoustic waveform; catching the one or more calculi or calculi fragments in the basket; and removing the basket with the one or more calculi or calculi fragments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a lithotripsy system including a device with an ultrasonic basket in an example.

FIG. 2 illustrates a close up view of a lithotripsy device with an ultrasonic basket in an example.

FIG. 3 illustrates a close up view of a lithotripsy device with an ultrasonic basket in an example.

FIG. 4 illustrates a method of removing calculi in an example.

### DETAILED DESCRIPTION

The present disclosure describes, among other things, a lithotripter device with a basket, used for applying both rotational and ultrasonic energy in the break-up and collection of a calculi. The shaft can act as a waveguide, propagating ultrasonic energy from a transducer to break up the calculus through vibration. The basket, at the distal end of the shaft, can rotate to physically collect broken up pieces of the calculus. The basket can also break up the calculus through vibrational energy. In one example, the basket itself can be made of a material that vibrates according to the waveform. In another example, the basket can host a series of mini-transducers thereon.

Some calculus masses, such as particularly hard calculi or large calculi in the kidney or ureter, can require a combination of techniques to address. For example, such calculi masses may need to both be dislodged and broken up. This can lead to a clinician having to use multiple different lithotripsy techniques to address the calculus mass in a single case, such as where a patient is afflicted with more challenging scenarios involving such hard calculi and large calculi.

Many lithotripsy devices can either be used to dislodge a calculus mass, or to break up a calculus mass. For example, in theory, a basket device can be used to extract a dislodged calculus. An ultrasonic device can, in contrast, be used to break up a larger calculus. In reality, basket devices are often used as a last step after a larger calculus has already been broken down into smaller fragments. In this case, the basket at the end of a catheter can be pushed over a guidewire that is set in place where the broken calculus is located. The use of multiple devices to accomplish dislodging and break up can take additional time. Moreover, this causes the surgeon to go in and out of the patient with multiple devices, causing additional risks in the procedure.

Discussed herein, a lithotripsy device can deliver both rotational energy and ultrasonic energy to a basket to generate vibrations. The device can be used to dislodge and break-up a calculus and pull it into the basket. In an example, the ultrasonic energy can be delivered to the basket through an external transducer device connected to the shaft of the device including the basket. In another example, localized transducers at the tip of the basket can be used which vibrate at ultrasonic frequencies to dislodge the calculus at the treatment site.

Additionally, in some cases, the basket can be rotated at a nominal rotational speed to dislodge the calculus effectively at the treatment site. The rotation can be accomplished along the shaft of the device, such as by an external rotation device such as a motor.

The discussed devices and methods herein have several advantages. For example, the use of a single device providing both ultrasonic energy and a basket can allow for a single device for dislodging and breaking up calculi. This can reduce the complexity of the lithotripsy procedure, while still addressing calculi that are very tightly entrenched in the kidney or ureter. The use of such a device can reduce the overall time of calculi ablation and removal with a lower number of incisions in the patient.

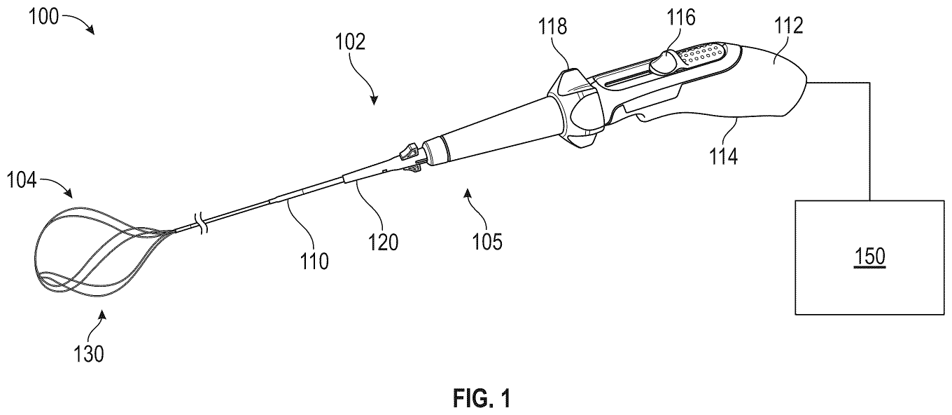

FIG. 1 illustrates a lithotripsy system 100 including a device 105 with an ultrasonic basket 130 in an example. The device 105 can be, for example, an ultrasonic probe with the basket 130. The device 105 can include a shaft 110 with a proximal portion 102 and a distal portion 104. The shaft 110 can extend from the handle portion 112 towards the basket 130. The handle portion 112 can include a grip 114, a basket opening mechanism 116, and a rotational mechanism 118. In the example of FIG. 1, a transducer 120 can be located on the proximal portion 102 of the shaft 110.

The lithotripsy system 100 can allow for effective calculi dislodgement and removal. In some cases, the device 105 can allow for dislodging and removal of a calculus without excessive fiber dust and fragmentation. In some cases, the device 105 can help contain the dusting area when an ultrasonic method is used to fragment the calculi. For example the device 105 can help collect fragments of the calculi in the basket 130 when ultrasonic (or other acoustic) energy is used to fragment the calculi.

The device 105 can be an elongated probe shaped and sized for insertion into a patient, such as for a lithotripsy procedure. The device 105 can be, for example, for use with an endoscope. The device 105 can be coupled to the controller 150 and the transducer 120, which can provide acoustic energy to the device 105 for fragmentation of calculi. At least one of the longitudinal shaft 110 or the basket 130 can be configured to vibrate in response to the acoustic energy input.

The shaft 110 can be a longitudinal shaft extending between the proximal portion 102 and the distal portion 104. The shaft 110 can be a waveguide along which acoustic energy from the transducer 120 can be propagated. The shaft 110 can have an attachment point with the transducer 120. The distal portion 104 of the shaft 110 can be inserted into a patient into contact with a target, such as a calculus, so as to allow the device 105 to use acoustic waves to fracture the target. In an example, the longitudinal shaft 110 can be nitinol or titanium, or another appropriate material that can serve as a waveguide.

The handle portion 112 can extend proximally from the shaft 110. The handle portion 112 can be for the user to hold and operate the device 105. The handle portion 112 can optionally include user controls, such as the basket opening mechanism 116 and the rotational mechanism 118, among other optional controls. The shaft 110 can extend proximally forward, such as in a cantilever fashion, from the handle portion 112. The handle portion 112 can allow for easy set-up of the system 100. The handle portion 112 can allow for operator actuation of the rotational mechanism 118 to rotate the basket 130 and the shaft 110, and operator actuation of the transducer 120 for provision of vibrational energy to the shaft 110, the basket 130, or both. The handle portion 112 can allow for efficient retrieval of calculi and calculi fragments.

The grip 114 can be on or integral to the handle portion 112. The grip 114 can be sized and shaped to fit an operator hand. The grip 114 can optionally include texture or shapes to aid in an operator holding the device 105. The basket opening mechanism 116 can be situated such that an operator can reach the basket opening mechanism 116 while holding the device 105. The basket opening mechanism 116 can be, for example, a lever, button, or other appropriate mechanism to help open and close the basket 130. The basket opening mechanism 116 can be operably coupled to the basket 130 down the length of the shaft 110.

The rotational mechanism 118 can be situated on or near the handle portion 112, such as at a proximal portion 102 of the shaft 110. The rotational mechanism 118 can be, for example, a rotational driver operably coupled to the longitudinal shaft and the basket. The rotational mechanism 118 can be actuatable for rotating the longitudinal shaft 110 and the basket 130. In an example, the rotational mechanism 118 can be manual, such as a rotational wheel. In another example, the rotational mechanism 118 can be automated. For example, a rotational motor can be used for the rotational mechanism 118. In an example, a smaller RPM driver could be used, such as actuatable with a button or trigger.

The transducer 120 can be an acoustic transducer that is acoustically coupled to the longitudinal shaft 110, the basket 130, or both. In an example, the acoustic transducer can be configured to propagate a waveform of about 20 kHz to about 150 kHz down the longitudinal shaft 110. The transducer 120 can be operably connected to an acoustic generator actuatable for producing an acoustic waveform. The shaft 110 waveguide can be configured to be moved or vibrated by the transducer 120.

For example, the transducer 120 can be an ultrasonic transducer that converts electrical energy to mechanical waves through the piezoelectric effect. The transducer 120 can include one or more piezoelectric members, such as a stack. Here, the piezoelectric members can be configured to receive a drive signal via the controller 150 to actuate the transducer 120. The piezoelectric effect can increase the mechanical length of the transducer 120 in response to a voltage on the transducer 120, such as provided by a generator. The change in length of the transducer 120 can be proportional to one or more variables including, but not limited to, the voltage level and the frequency in which the signal is applied to the transducer 120. When the electrical frequency applied to the transducer is equal to the time for the mechanical wave to traverse the crystal and return, optimal energy conversion may occur due to resonance and can create a mechanical displacement that is many times larger than at any other frequencies. In some cases, the transducer 120 can be used to produce a variety of different waveforms, such as providing harmonic driving opportunities for driving the transducer 120 at different frequencies to affect the displacement of the waveguide.

The transducer 120 can be used to propagate a variety of waveforms down the shaft 110 and/or to the basket 130. For example, sinusoid waveforms or generally square waveforms can be used. Such waveforms propagated down the device 105 can cause vibrational movement at the distal portion 104 of the shaft 110, such as at the basket 130. The vibrational movement can help to dislodge calculi, break down calculi, or both.

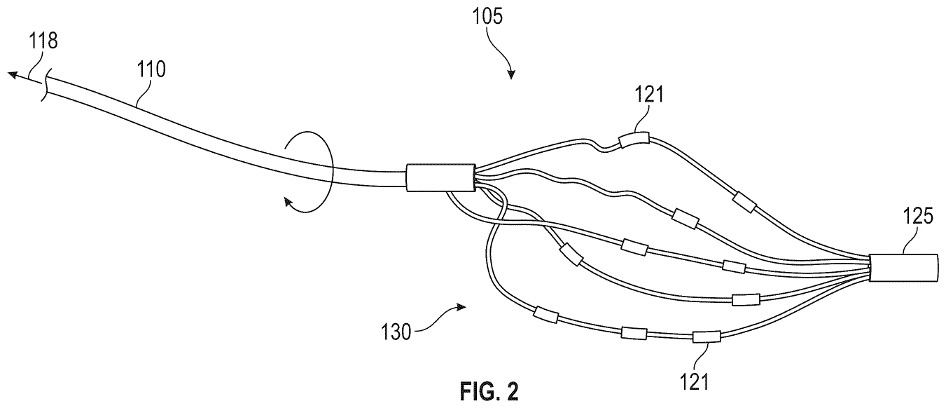

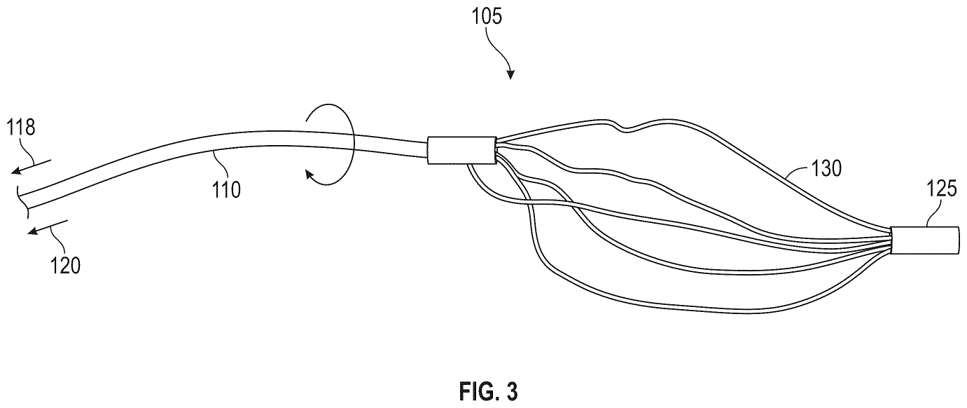

The basket 130 can extend along the distal portion 104 of the shaft 110. The basket 130 can be sized and shaped to help catch calculi, calculi fragments, or both, therein. In an example, the basket 130 can include a bullet tip or wire-guided design. The basket 130 can include an end piece 125 at the proximal end of the basket 130, as illustrated in FIGS. 2 and 3. The end piece 125 can be situated where various prongs of the basket 130 converge.

The basket 130 can be configured to rotate along with rotational movement of the rotational mechanism 118. The basket 130 itself can rotate along with the shaft 110 when the rotational mechanism 118 is actuated. This can aid in dislodging a collection of calculi and calculi fragments. For example, the basket 130 can rotate proximal of the end piece 125.

The basket 130 can be configured to vibrate along with an acoustic waveform produced by the transducer 120. For example, the basket 130 itself can be acoustically-transmissive. In an example, the basket 130 can be made of nitinol, titanium, or another appropriate material that is acoustically-transmissive. In some cases, end piece 125 can be configured to vibrate according to the waveform. In some cases, transducers can be situated on the basket 130 itself to provide the vibrational energy thereon.

The controller 150 can be used to actuate and control the lithotripsy system 100. The controller can include a driver, or control a driver, to send drive signals to the transducer 120. In an example, the controller 150 can be integrated with a generator for provision of voltage to the transducer 120 and production of the waveform.

The controller 150 can include a processor (which may be referred to as a central processor unit or CPU) that is in communication with memory devices including a secondary storage, read only memory (ROM), random access memory (RAM), input/output (I/O) devices, and network connectivity devices, as needed. The processor can be implemented as one or more CPU chips or may be part of one or more application specific integrated circuits (ASICs).

The device described herein may be configured to include computer-readable non-transitory media storing computer readable instructions and one or more processors coupled to the memory, and when executing the computer readable instructions configure the controller to perform method steps and operations described above with reference to the above figures. The computer-readable non-transitory media includes all types of computer readable media, including magnetic storage media, optical storage media, flash media and solid-state storage media.

FIG. 2 and FIG. 3 show close up views of the device 105, with different mechanisms for vibration at the target site for removal of calculi. FIG. 2 illustrates a close up view of the lithotripsy device 105 with a basket 130 having multiple transducers 121 in an example. Here, the shaft 110 with the basket 130 and the end piece 125 can rotate upon actuation of the rotational mechanism 118. As shown in FIG. 2, multiple transducers 121 can be situated on and in the basket 130. The transducers 121 can be actuatable to vibrate and help dislodge or break-up calculi.

The basket 130 with the transducers 121 can be situated by the operator at the target surgical site, such as near one or more calculi of interest. The rotation of the basket 130 can be used to dislodge the calculus, and/or break-up the calculus. The vibration of the transducers 121 can also be used to dislodge and/or break-up calculi depending on the specific energy provided to the transducers 121. The transducers 121 can vibrate according to energy such as an acoustic waveform provided down the shaft 110 to the transducers 121, such as from a generator. Dislodged calculi and fragments can fall into and be collected within the basket 130, such as when the basket 130 is rotated by the rotational mechanism 118. The vibrations of the transducers 121 can be, for example, ultrasonic vibration in the range of about 20 kHz to about 150 kHz.

FIG. 3 illustrates a close up view of the lithotripsy device 105 with an ultrasonic basket 130 in an example. In this case, the basket 130 can be made of a material, such as titanium or nitinol, that can vibrate according to acoustic energy provided by the transducer 120. The shaft 110 and the basket 130 can be rotated such as by actuation of the rotation mechanism 118. Additionally, when the transducer 120 provides a waveform down the shaft 110, both the shaft 110 and the basket 130 itself can vibrate accordingly.

The basket 130 itself can be responsive to acoustic waveform movement. The waveform can propagate through the end piece 125 in some cases, causing the entirety of the basket 130 to vibrate and aid in removal of calculi. In either the case of FIG. 2 and FIG. 3, localized acoustic energy can be used to help breakup and remove calculi.

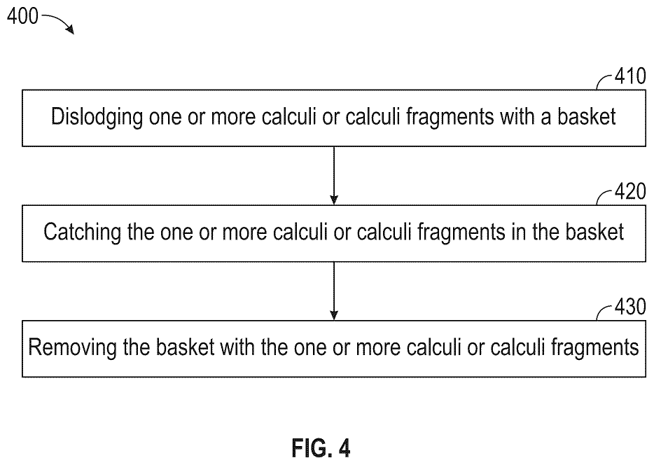

FIG. 4 illustrates a method 400 of removing calculi in an example. At step 410, an operator can dislodge one or more calculi or calculi fragments with a basket. Dislodging can include vibrating the basket according to an acoustic waveform. Vibrating the basket can include actuating one or more acoustic transducers on the basket. In some cases, this step can also include fragmenting the one or more calculi or calculi fragments.

At step 420, the operator can catch the one or more calculi or calculi fragments in the basket, such as by rotational movement. At step 430, the basket with the one or more calculi or calculi fragments can be removed from the patient.

### VARIOUS NOTES & EXAMPLES

In some aspects, the techniques described herein relate to a lithotripsy device including: a longitudinal shaft extending between a proximal portion and distal portion; a basket extending along the distal portion of the longitudinal shaft, the basket configured to catch a target calculus; and a rotational driver operably coupled to the longitudinal shaft and the basket, the rotational driver actuatable for rotating the longitudinal shaft and the basket, wherein at least one of the longitudinal shaft and the basket is configured to vibrate in response to an acoustic energy input.

In some aspects, the techniques described herein relate to a lithotripsy device, further including at least one acoustic transducer that is acoustically coupled to at least one of the longitudinal shaft or the basket.

In some aspects, the techniques described herein relate to a lithotripsy device, further including an acoustic generator operably coupled to the acoustic transducer, the acoustic generator actuatable for producing an acoustic waveform.

In some aspects, the techniques described herein relate to a lithotripsy device, wherein the basket is acoustically-transmissive.

In some aspects, the techniques described herein relate to a lithotripsy device, wherein the basket includes nitinol or titanium.

In some aspects, the techniques described herein relate to a lithotripsy device, wherein the basket includes a plurality of acoustic transducers thereon.

In some aspects, the techniques described herein relate to a lithotripsy device, wherein the acoustic transducer is configured to propagate a waveform of about 20 kHz to about 150 kHz down the longitudinal shaft.

In some aspects, the techniques described herein relate to a lithotripsy device, wherein the longitudinal shaft includes nitinol or titanium.

In some aspects, the techniques described herein relate to a lithotripsy device, wherein the rotational driver is manual.

In some aspects, the techniques described herein relate to a lithotripsy device, wherein the rotational driver is automated.

In some aspects, the techniques described herein relate to a lithotripsy device, wherein the basket includes a proximal end configured to vibrate according to the waveform.

In some aspects, the techniques described herein relate to a lithotripsy device, wherein the basket is configured to vibrate with the waveform.

In some aspects, the techniques described herein relate to a lithotripsy device, wherein the longitudinal shaft is configured to vibrate with the waveform.

In some aspects, the techniques described herein relate to an ultrasonic probe including: a longitudinal shaft extending between a proximal portion and distal portion; a basket extending along the distal portion of the longitudinal shaft, the basket configured to catch a target calculus; a rotational driver operably coupled to the longitudinal shaft and the basket, the rotational driver actuatable for rotating the longitudinal shaft and the basket; and a transducer acoustically coupled to at least one of the longitudinal shaft or the basket, the transducer configured to produce an ultrasonic waveform, wherein the basket is configured to vibrate in response to an acoustic energy input.

In some aspects, the techniques described herein relate to a probe, wherein the transducer is on the shaft.

In some aspects, the techniques described herein relate to a probe, wherein the longitudinal shaft is configured to vibrate with the waveform.

In some aspects, the techniques described herein relate to a probe, wherein the transducer includes a plurality of transducers on the basket.

In some aspects, the techniques described herein relate to a method of removing a calculus, the method including: dislodging one or more calculi or calculi fragments with a basket, wherein dislodging includes vibrating the basket according to an acoustic waveform; catching the one or more calculi or calculi fragments in the basket; and removing the basket with the one or more calculi or calculi fragments.

In some aspects, the techniques described herein relate to a method, further including fragmenting the one or more calculi or calculi fragments.

In some aspects, the techniques described herein relate to a method, wherein vibrating the basket includes actuating one or more acoustic transducers on the basket.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A lithotripsy device comprising:
a longitudinal shaft extending between a proximal portion and distal portion;
a basket extending along the distal portion of the longitudinal shaft, the basket configured to catch a target calculus; and
a rotational driver operably coupled to the longitudinal shaft and the basket, the rotational driver actuatable for rotating the longitudinal shaft and the basket,
wherein at least one of the longitudinal shaft and the basket is configured to vibrate in response to an acoustic energy input.

2. The lithotripsy device of claim 1, further comprising at least one acoustic transducer that is acoustically coupled to at least one of the longitudinal shaft or the basket.

3. The lithotripsy device of claim 2, further comprising an acoustic generator operably coupled to the acoustic transducer, the acoustic generator actuatable for producing an acoustic waveform.

4. The lithotripsy device of claim 1, wherein the basket is acoustically-transmissive.

5. The lithotripsy device of claim 1, wherein the basket comprises nitinol or titanium.

6. The lithotripsy device of claim 1, wherein the basket comprises a plurality of acoustic transducers thereon.

7. The lithotripsy device of claim 2, wherein the acoustic transducer is configured to propagate a waveform of about 20 kHz to about 150 kHz down the longitudinal shaft.

8. The lithotripsy device of claim 1, wherein the longitudinal shaft comprises nitinol or titanium.

9. The lithotripsy device of claim 1, wherein the rotational driver is manual.

10. The lithotripsy device of claim 1, wherein the rotational driver is automated.

11. The lithotripsy device of claim 1, wherein the basket comprises a proximal end configured to vibrate according to a waveform.

12. The lithotripsy device of claim 1, wherein the basket is configured to vibrate with a waveform.

13. The lithotripsy device of claim 1, wherein the longitudinal shaft is configured to vibrate with a waveform.

14. An ultrasonic probe comprising:
a longitudinal shaft extending between a proximal portion and distal portion;
a basket extending along the distal portion of the longitudinal shaft, the basket configured to catch a target calculus;
a rotational driver operably coupled to the longitudinal shaft and the basket, the rotational driver actuatable for rotating the longitudinal shaft and the basket; and
a transducer acoustically coupled to at least one of the longitudinal shaft or the basket, the transducer configured to produce an ultrasonic waveform,
wherein the basket is configured to vibrate in response to an acoustic energy input.

15. The probe of claim 14, wherein the transducer is on the shaft.

16. The probe of claim 14, wherein the longitudinal shaft is configured to vibrate with the waveform.

17. The probe of claim 14, wherein the transducer comprises a plurality of transducers on the basket.

18. A method of removing a calculus, the method comprising:
dislodging one or more calculi or calculi fragments with a basket, wherein dislodging comprises vibrating the basket according to an acoustic waveform;
catching the one or more calculi or calculi fragments in the basket; and
removing the basket with the one or more calculi or calculi fragments.

19. The method of claim 18, further comprising fragmenting the one or more calculi or calculi fragments.

20. The method of claim 18, wherein vibrating the basket comprises actuating one or more acoustic transducers on the basket.
